(19) United States
(12) Patent Application Publication
(10) Pub. No.: US 2011/0232159 A1
(43) Pub. Date: Sep. 29, 2011

(54) Biodiesel Additive

(75) Inventors: Andrew Westlake, Warrambool (AU); Stewart McGlashan, Annerley (AU); Stephen Clarke, Morphett Vale (AU); Mark Fisher, Glenelg East (AU); Rachel Pillar, Edwardstown (AU); Kristina Constantopoulos, Malvern (AU); Simon Mathew, Malvern (AU); Elda Markovic, Morphettiville (AU); Eleni Papadopoulos, St. Marys (AU); David John Clarke, Chartwell (AU); Kim Anh-Thi Nguyen, Mawsons Lake (AU)

(73) Assignee: Meat & Livestock Australia Limited, North Sydney (AU)

(21) Appl. No.: 12/994,771

(22) PCT Filed: May 26, 2009
(86) PCT No.: PCT/AU2009/000657

§ 371 Date: Jun. 9, 2011

(30) Foreign Application Priority Data
May 26, 2008 (AU) 2008902610

(51) Int. Cl. C10L 1/19; C07H 13/02

(52) U.S. Cl. 44/307; 44/313; 536/115

(57) ABSTRACT

The invention provides an additive for lowering the minimum usable temperature of a biodiesel fuel or a diesel/biodiesel blend. The additive comprises at least one saccharide ester and a polymer having a comb structure. In some instances there is only one saccharide ester present. In this case the saccharide ester comprises at least one saturated ester group and at least one unsaturated ester group. In other instances, there is more than one saccharide ester is present. In these cases the additive comprises a first saccharide ester comprising at least one saturated ester group and a second saccharide ester comprising at least one unsaturated ester group.

### TECHNICAL FIELD

The present invention relates to an additive for a biodiesel fuel or for a diesel/biodiesel blend, and to a biodiesel fuel or diesel/biodiesel blend containing the additive.

### BACKGROUND OF THE INVENTION

Biodiesel is an alternative to petroleum diesel fuel and is made from renewable resources such as vegetable oils, animal fats or algae. It has very similar combustion properties to petroleum diesel and can replace it in many current uses. Biodiesel (FAME) has found extensive use as additive to ULSD (ultra-low sulfur petrodiesel fuel) as a lubricity agent, improving the otherwise low lubricity of pure ULSD. A growing number of fuel stations throughout Australia and around the world are making biodiesel and biodiesel blends available to consumers, and a growing number of large transportation fleets use some proportion of biodiesel in their fuel.

Biodiesel has environmental benefits in comparison to petroleum based fuels:
Biodiesel reduces emissions of carbon monoxide (CO) by about 50% and carbon dioxide by between about 50 and about 80% on a net basis because the carbon released in the biodiesel emission originates from fixation/sequestration of atmospheric CO₂ rather than carbon originating from sources within the earth's crust.
Use of biodiesel reduces emissions of oxides of sulfur (SOₓ), because biodiesel contains very low level of sulfur.
Biodiesel reduces by as much as 65% the emission of particulates (small particles of solid combustion products).
Biodiesel does produce more nitrogen oxide (NOx) emissions than petrodiesel, but these emissions can be reduced through the use of catalytic converters and tallow.
Biodiesel has a higher cetane rating than petrodiesel and therefore causes less knocking.

In spite of biodiesel's many advantages, performance during cold weather may affect its year-round commercial viability in moderate temperature climates. Biodiesel made from feed stocks containing higher concentrations of high-melting point saturated long-chain fatty acids, (e.g. beef tallow, a triglyceride of fatty acids) tend to exhibit relatively poor cold flow properties. Myristic, palmitic and stearic acid are the major saturated fatty acids while palmitoleic, oleic and linoleic are the major unsaturated fatty acids. Saturated fatty acid composition ranges from 47-55% whereas the unsaturated fatty acid composition ranges from 43-52%. Due to its content of saturated compounds such as fatty acid methyl esters (FAMEs), in particular tallow methyl ester (TME), biodiesel generally has a cloud point ranging from about 5 to about 20° C. In addition, both fluids, biodiesel and petrodiesel, increase in viscosity with decreasing temperature. As a result, changes in viscosity restrict the flow through a vehicle's fuel handling system. In addition filter blockages from large wax crystals can occur.

Cold flow improver (CFI) additives have been developed in the past for treating petrodiesel fuel and reducing this problem. However it has been found that CFI additives structurally designed to modify paraffin-based crystal modification in petrodiesel and such activity do not act upon the FAME based nucleation observed in biodiesel. As a result there is a requirement for the development of new CFI additives for FAME based biodiesel that exhibits efficacy in the suppression of nucleation and crystal growth mechanisms observed in biodiesels containing high levels of saturated FAME.

### SUMMARY OF THE INVENTION

In a first aspect of the invention there is provided an additive for lowering the minimum usable temperature of a biodiesel fuel or a diesel/biodiesel blend, said additive comprising:
at least one saccharide ester, and
a polymer having a comb structure,
   wherein:
if only one saccharide ester is present, said saccharide ester comprises at least one saturated ester group and at least one unsaturated ester group; and
if more than one saccharide ester is present, these comprise a first saccharide ester comprising at least one saturated ester group and a second saccharide ester comprising at least one unsaturated ester group.

The following options may be used in the first aspect either individually or in any suitable combination.

A combination of the additive with the biodiesel fuel or diesel/biodiesel blend at less than about 3.5% of said additive may have a reduced cold filter plugging point relative to said biodiesel or diesel/biodiesel blend without said additive.

The biodiesel may be animal derived biodiesel. It may comprise tallow and/or lard. It may additionally comprise a plant derived biodiesel.

The ratio of the polymer to the saccharide ester may be between about 5:1 and about 20:1 w/w. The polymer may be a maleic anhydride derived copolymer. It may be a maleic anhydride/acrylate ester/methacrylate ester derived terpolymer. It may be a terpolymer of maleic anhydride an alkyl acrylate and an alkyl methacrylate. The polymer may comprise side chains derived from lauric acid, side chains derived from stearic acid or both of these. The polymer may comprise a copolymer of maleic anhydride, lauryl acrylate and stearyl methacrylate. The ratio of these may be about 1:1.5-2.5:0.4-0.6, e.g. about 1:2:0.5.

In some embodiments one saccharide ester is present, said saccharide ester comprising at least one saturated ester group and at least one unsaturated ester group. In other embodiments the additive comprises a first saccharide ester comprising at least one saturated ester group and a second saccharide ester comprising at least one unsaturated ester group. The first saturated ester group may have no unsaturated ester groups. It may have only saturated ester groups. The second saccharide ester may have no saturated ester groups. It may have only unsaturated ester groups.

The saccharide ester, or both the first and second saccharide esters, may have an average of about 5 ester groups per molecule. The saccharide ester, or at least one of the first and second saccharide esters, may be a disaccharide ester. The disaccharide ester may be a sucrose ester. The sucrose ester may be a sucrose myristate oleate mixed ester. If first and second saccharide esters are present, the first saccharide ester may be sucrose myristate and the second saccharide ester may be sucrose oleate.

The ratio of saturated esters (i.e. saturated ester groups) to unsaturated esters (i.e. unsaturated ester groups) may be about 2:1 on a weight or molar basis. The saturated esters and the unsaturated esters may be in the same molecule or may be in different molecules.

The additive may additionally comprise diesel and/or biodiesel. The proportion of diesel and/or biodiesel to polymer may be between about 0.5 and about 5 to 1.

In an embodiment there is provided an additive for lowering the minimum usable temperature of a biodiesel fuel or a diesel/biodiesel blend, said additive comprising:
a first saccharide ester comprising at least one saturated ester group and a second saccharide ester comprising at least one unsaturated ester group, and
a maleic anhydride derived copolymer.

In another embodiment there is provided an additive for lowering the minimum usable temperature of a biodiesel fuel or a diesel/biodiesel blend, said additive comprising:
sucrose myristate and sucrose oleate, and
a terpolymer of maleic anhydride an alkyl acrylate and an alkyl methacrylate.

In another embodiment the additive comprises:
(i) sucrose myristate;
(ii) sucrose oleate; and
(iii) a terpolymer of maleic anhydride, lauryl acrylate and stearyl methacrylate in a molar ratio of about 1:2:0.5;
   wherein the ratio of (i):(ii) is about 2:1 on a molar basis and the ratio of (i)+(ii):(iii) is about 1:10 on a weight basis.

In a second aspect of the invention there is provided a biodiesel or diesel/biodiesel blend comprising an additive according to the first aspect (optionally including any one or more of the options specified).

The additive may be present in an amount sufficient to reduce the cold filter plugging point of the biodiesel or diesel/biodiesel blend. It may be present in an amount sufficient to reduce the cold filter plugging point of the biodiesel or diesel/biodiesel blend by at least 2° C. The additive may be present in the biodiesel or diesel/biodiesel blend at less than about 3.5%, or between about 0.1 and about 3.5% w/w. It may be present in the biodiesel fuel or diesel/biodiesel blend at less than about 3.5% and the cold filter plugging point of said biodiesel or diesel/biodiesel blend may be reduced by at least about 2° C. relative to said biodiesel or diesel/biodiesel blend without said additive.

The biodiesel or diesel/biodiesel blend may be substantially homogeneous. It may be substantially homogeneous at about 20° C. There may be no suspended solids therein. There may be no suspended solids therein at about 20° C.

In an embodiment there is provided a biodiesel fuel or diesel/biodiesel blend comprising:
a biodiesel or mixture of diesel and biodiesel;
at least one saccharide ester, and
a polymer having a comb structure,
   wherein:
if only one saccharide ester is present, said saccharide ester comprises at least one saturated ester group and at least one unsaturated ester group; and
if more than one saccharide ester is present, these comprise a first saccharide ester comprising at least one saturated ester group and a second saccharide ester comprising at least one unsaturated ester group.

In another embodiment there is provided a biodiesel fuel or diesel/biodiesel blend comprising:
(i) a biodiesel or mixture of diesel and biodiesel
(ii) sucrose myristate;
(iii) sucrose oleate; and
(iv) a terpolymer of maleic anhydride, lauryl acrylate and stearyl methacrylate in a molar ratio of about 1:2:0.5;
   wherein the ratio of (ii):(iii) is about 2:1 on a molar basis and the ratio of (ii)+(iii):(iv) is about 1:10 on a weight basis and wherein the biodiesel fuel or diesel/biodiesel blend is substantially homogeneous at about 20° C.

In a third aspect of the invention there is provided a process for preparing an additive for lowering the minimum usable temperature a biodiesel fuel or a diesel/biodiesel blend, said process comprising combining a polymer having a comb structure and at least one saccharide ester, wherein:
if only one saccharide ester is used, said saccharide ester comprises at least one saturated ester group and at least one unsaturated ester group; and
if more than one saccharide ester is used, these comprise a first saccharide ester comprising at least one saturated ester group and a second saccharide ester comprising at least one unsaturated ester group.

The polymer and the at least one saccharide ester may be combined in a ratio of between about 5:1 and about 20:1 w/w. The process may comprise mixing diesel or biodiesel with the polymer prior to said combining. The process may comprise mixing the diesel or biodiesel with the at least one saccharide ester prior to said combining. The process may comprise mixing the combined polymer and at least one saccharide ester with diesel or biodiesel.

In some embodiments one saccharide ester is used, said saccharide ester comprising at least one saturated ester group and at least one unsaturated ester group. In other embodiments the process uses a first saccharide ester comprising at least one saturated ester group and a second saccharide ester comprising at least one unsaturated ester group. The first saturated ester group may have no unsaturated ester groups. It may have only saturated ester groups. The second saccharide ester may have no saturated ester groups. It may have only unsaturated ester groups.

The saccharide ester, or both the first and second saccharide esters, may have an average of about 5 ester groups per molecule. The saccharide ester, or at least one of the first and second saccharide esters, may be a disaccharide ester. The disaccharide ester may be a sucrose ester. The sucrose ester may be a sucrose myristate oleate mixed ester. If first and second saccharide esters are used, the first saccharide ester may be sucrose myristate and the second saccharide ester may be sucrose oleate.

The polymer may be a maleic anhydride derived copolymer. It may be a maleic anhydride/acrylate ester/methacrylate ester derived terpolymer. It may be a terpolymer of maleic anhydride an alkyl acrylate and an alkyl methacrylate. The polymer may comprise side chains derived from lauric acid, side chains derived from stearic acid or both of these.

In an embodiment there is provided a process for preparing an additive for a biodiesel fuel or for a diesel/biodiesel blend, said process comprising combining:
sucrose myristate;
sucrose oleate; and
a terpolymer of maleic anhydride, lauryl acrylate and stearyl methacrylate in a molar ratio of about 1:2:0.5.

In another embodiment there is provided a process for preparing an additive for a biodiesel fuel or for a diesel/biodiesel blend, said process comprising combining:
sucrose myristate;
sucrose oleate; and
a terpolymer of maleic anhydride, lauryl acrylate and stearyl methacrylate in a molar ratio of about 1:2:0.5;
   to form a mixture, and then homogenising the mixture, wherein the sucrose myristate and the sucrose oleate are in a molar ratio of about 2:1 and each has an average of about 5 ester groups per molecule.

The invention also provides an additive for a biodiesel fuel or for a diesel/biodiesel blend when made by the process of the third aspect.

In a fourth aspect of the invention there is provided a method for reducing the minimum usable temperature of a biodiesel fuel or of a diesel/biodiesel blend, said method comprising combining said biodiesel fuel or diesel/biodiesel blend with an additive according to the first aspect, or made by the process of the third aspect.

The additive may be combined with the biodiesel fuel or diesel/biodiesel blend at a ratio of between about 0.1 and 3.5% w/w.

The invention also provides a process for making a biodiesel fuel or diesel/biodiesel blend having a reduced minimum usable temperature, said process comprising combining a biodiesel fuel or diesel/biodiesel blend with an additive according to the first aspect, or made by the process of the third aspect. The invention also provides a biodiesel fuel or diesel/biodiesel blend having a lowered minimum usable temperature, said biodiesel fuel or diesel/biodiesel blend being made by this process.

In a fifth aspect of the invention there is provided the use of at least one saccharide in lowering the minimum usable temperature of a biodiesel fuel or of a diesel/biodiesel blend, wherein:
if only one saccharide ester is used, said saccharide ester comprises at least one saturated ester group and at least one unsaturated ester group; and
if more than one saccharide ester is used, these comprise a first saccharide ester comprising at least one saturated ester group and a second saccharide ester comprising at least one unsaturated ester group.

In an embodiment there is provided the use of a mixture of sucrose myristate and sucrose oleate in a molar ratio of about 2:1, each having an average of about 5 ester is groups per molecule, in reducing the minimum usable temperature of a biodiesel fuel or of a diesel/biodiesel blend.

In a sixth aspect of the invention there is provided the use of at least one saccharide in the manufacture of an additive for lowering the minimum usable temperature of a biodiesel fuel or a diesel/biodiesel blend, wherein:
if only one saccharide ester is used, said saccharide ester comprises at least one saturated ester group and at least one unsaturated ester group; and
if more than one saccharide ester is used, these comprise a first saccharide ester comprising at least one saturated ester group and a second saccharide ester comprising at least one unsaturated ester group.

In an embodiment there is provided the use of a mixture of sucrose myristate and sucrose oleate in a molar ratio of about 2:1, each having an average of about 5 ester groups per molecule, in the manufacture of an additive for a biodiesel fuel or for a diesel/biodiesel blend, said additive being capable of lowering the minimum usable temperature of a biodiesel fuel or of a diesel/biodiesel blend.

### DETAILED DESCRIPTION OF THE INVENTION

The present invention describes an additive for a biodiesel fuel or for a diesel/biodiesel blend. The additive comprises at least one saccharide ester. Saccharide esters are capable of performing as surfactants, and may therefore serve to compatiblise other components of the additive, e.g. a polymer, or to compatiblise, solubilise, disperse or emulsify those components with the biodiesel fuel or diesel/biodiesel blend. Thus the resulting additive may be a solution, an emulsion, a microemulsion, a dispersion etc. or it may be more than one of these.

Where reference is made to “a saccharide ester”, this may refer to a mixture of saccharide esters which vary in the degree of esterification and/or in the regiochemistry of the esterification. Thus for example “sucrose oleate” refers to a mixture of sucrose trioleate, sucrose tetraoleate, sucrose pentaoleate etc. Also, in sucrose trioleate, for example, there are a number of structural isomers, which differ in the carbon atoms of the sucrose nucleus to which the oleate groups are attached.

If only one saccharide ester is used, said saccharide ester comprises at least one saturated ester group and at least one unsaturated ester group. Thus in this case the saccharide ester is a saccharide mixed ester (i.e. has more than one different ester group per molecule). If more than one saccharide ester is used, these comprise a first saccharide ester comprising at least one saturated ester group and a second saccharide ester comprising at least one unsaturated ester group. In this case the first and second saccharide esters may each, independently, be either saccharide mixed esters or saccharide homoesters (i.e. have only one type of ester group per molecule).

Ester groups are of the general formula RC(═O)O— where R is an optionally substituted hydrocarbon group. In the context of this specification, a saturated ester group is an ester group in which R has no C═C or C≡C bonds, and an unsaturated ester group is one in which R has one or more C═C or C≡C bonds. Thus in saturated ester groups, R is an optionally substituted alkyl group, whereas in unsaturated ester groups R is an optionally substituted alkenyl group or alkynyl group. The unsaturated ester groups may have more than one double bond. They may have for example 1, 2, 3, 4 or 5 double bonds. They may be monounsaturated or may be polyunsaturated. The saturated ester groups may have a degree of unsaturation of 1 (from the C═O of the ester group). The unsaturated ester groups may have a degree of unsaturation greater than 1, e.g. 2 to 6, or 2 to 4 or 3 to 6, for example 2, 3, 4, 5 or 6.

A combination of the additive with the biodiesel fuel or diesel/biodiesel blend at less than about 3.5% of said additive may have a cold filter plugging point (CFPP) which is reduced by at least about 2° C. relative to said biodiesel or diesel/biodiesel blend without said additive. The actual lowering of CFPP may depend on the precise nature of the additive, on the nature of the biodiesel fuel (e.g. its biological origin, its chemical makeup etc.), the ratio of the biodiesel to the diesel in the case of a blend, the nature of any other additives in the biodiesel or blend etc. The additive may, for example, be used in the biodiesel or blend at between about 0.1 and about 3.5% w/w or w/v or about 0.1 to 3.5, 0.1 to 2, 0.1 to 1, 0.1 to 0.5, 0.5 to 3.5, 1 to 3.5, 0.5 to 1 or 1 to 2%, e.g. about 0.1, 0.2, 0.3, 0.4, 0.5, 0.6, 0.7, 0.8, 0.9, 1, 1.25, 1.5, 1.75, 2, 2.5, 3 or 3.5% w/w or w/v. The resulting lowering of CFPP may be between about 1 and 10° C., or about 1 to 5, 1 to 2, 2 to 10, 5 to 10 or 2 to 5° C., e.g. about 1, 2, 3, 4, 5, 6, 7, 8, 9 or 10° C., although in some cases it may be greater than this. In a similar manner, cloud point (CP) may be reduced by between about 1 and 10° C., or about 1 to 5, 1 to 2, 2 to 10, 5 to 10 or 2 to 5° C., e.g. about 1, 2, 3, 4, 5, 6, 7, 8, 9 or 10° C., although in some cases it may be greater than this.

The present invention may be used in conjunction with any suitable biodiesel or biodiesel blend. It may be used in conjunction with a biodiesel or biodiesel blend wherein said biodiesel or blend comprises a FAME. Commonly it is used in conjunction with animal or vegetable derived biodiesel or a blend of the two, as these materials are commonly in need of reduction in CFPP and CP. The biodiesel may comprise at least one of tallow and lard. It may be derived for example from beef, mutton, lamb, chicken, ox, pork, buffalo or some other suitable animal source or vegetable source. It may comprise biodiesel derived from palm (e.g. high light palm oil) or canola. Suitable biodiesel blends may have a biodiesel content of about 2 to about 95% by weight or volume, or about 2 to 50, 2 to 20, 2 to 10, 10 to 95, 20 to 95, 50 to 95, 10 to 50 or 20 to 50%, e.g. about 5, 10, 15, 20, 25, 30, 35, 40, 45, 50, 55, 60, 65, 70, 75, 80, 85, 90 or 95%. The blend may comprise a conventional diesel fuel. It may comprise petrol. It may comprise a hydrocarbon liquid. It may also comprise some vegetable derived biodiesel, e.g. canola derived biodiesel.

The additive may additionally comprise a polymer. The polymer may be capable of lowering the cold flow plugging point of a biodiesel, or of a biodiesel/diesel blend. The polymer may have a comb structure, in which side chains are attached to the backbone chain. The ratio of the polymer to the at least one saccharide ester in the additive may be between about 5:1 and about 20:1 w/w, or about 5:1 to 10:1, 10:1 to 20:1 or 8:1 to 15:1, e.g. about 5:1, 6:1, 7:1, 8:1, 9:1, 10:1, 11:1, 12:1, 13:1, 14:1, 15:1, 16:1, 17:1, 18:1, 19:1 or 20:1.

The polymer may be a copolymer. It may be a random copolymer, an alternating copolymer, a block copolymer or may have some other architecture. It may be a binary copolymer, a terpolymer or may have more than 3 types of monomer unit. The polymer may be a maleic anhydride derived copolymer. It may comprise maleic anhydride derived units. In one example the polymer is a maleic anhydride-alkyl acrylate-alkyl methacrylate terpolymer. The alkyl groups of the alkyl acrylate and the alkyl methacrylate may, independently, be straight chain alkyl groups or branched chain alkyl groups and may comprise cyclic structures. They may be hydrocarbon groups or may be substituted hydrocarbon groups. They may be saturated or may be unsaturated or some may be saturated and others unsaturated. They may, independently, have about 12 to about 24 carbon atoms, or about 12 to 18, 12 to 16, 14 to 24, 16 to 24, 20 to 24, 14 to 18, 14 to 16 or 16 to 18, e.g. about 12, 13, 14, 15, 16, 17, 18, 19, 20, 21, 22, 23 or 24 carbon atoms. They may have a mixture of chain lengths centred around any of these values. The polymer may for example comprise side chains derived from lauric acid, side chains derived from stearic acid or both of these. An example of a suitable polymer is a random terpolymer of maleic anhydride (MA), lauryl acrylate (LA) and stearyl methacrylate (SM). The ratio of these may be about 1:1.5-2.5:0.4-0.6. The amount of LA relative to MA may be about 1.5 to 2, 2 to 2.5 or 1.8 to 2.2, e.g. about 1.5, 1.6, 1.7, 1.8, 1.9, 2, 2.1, 2.2, 2.3, 2.4 or 2.5, and the amount of SM relative to MA may be about 0.4 to 0.5, 0.5 to 0.6 or 0.45 to 0.55, e.g. about 0.4, 0.45, 0.5, 0.55 or 0.6. A suitable ratio of monomer units in the polymer may be about 1:2:0.5.

The saccharide ester, or each ester independently, may be a monosaccharide ester or may be a disaccharide ester. Each, independently, may be a sucrose ester. The ester group, or each ester group independently, may have about 12 to about 20 carbon atoms, or about 12 to 16, 16 to 20 or 14 to 18 carbon atoms, e.g. about 12, 13, 14, 15, 16, 17, 18, 19 or 20 carbon atoms. It (or they, independently) may be saturated or unsaturated. The sucrose ester may be capable of acting as a surfactant. It may for example comprise sucrose myristate (SM) or sucrose oleate (SO) or a mixture of these. The ratio of these may be between about 3:1 and about 1:1, or about 3:1 and 1.5:1 or 1:5:1 and 1:1, e.g. about 3:1, 2.5:1, 2:1, 1.5:1 or 1:1. Each of the saccharide esters may be esterified to a degree of between about 50 and about 100% on a molar basis, or about 50 to 70, 60 to 90, 70 to 100, 80 to 100, 60 to 80 or 80 to 90%, e.g. about 50, 55, 60, 65, 70, 75, 80, 85, 90, 95 or 100%. In the case of a sucrose ester, each molecule may have an average of between about 3 and about 7, or about 4 to 6, 4 to 5, 5 to 6 or 4.5 to 5.5, e.g. about 3, 3.5, 4, 4.5, 5, 5.5, 6, 6.5 or 7 ester groups per molecule.

The additive may additionally comprise diesel or biodiesel. Thus the saccharide ester(s) and polymer may be mixed, optionally homogenised or dissolved in, diesel or biodiesel. This may serve to facilitate mixing of the additive with diesel or biodiesel when adding the additive to the diesel or biodiesel.

The exact composition of the additive, including for example such factors as the length of the side chains on the saccharide ester(s) and the polymer, the ratio of these, any other components, the degree of esterification of the saccharide ester, may be tailored so to optimise its effect in regard to any particular biodiesel or blend in which the additive is to be used. Thus for example the length of a carbon chain in an ester group of the saccharide ester and/or a length of a side chain of the polymer (if present) may be matched to the length of a carbon chain in the biodiesel. In this context the term “matched” does not necessarily indicate that the chain length will be the same as that in the biodiesel, but rather indicates that it will be optimised for use in the biodiesel or blend, so as, for example, to minimise CP and/or CFPP.

The additive may be present in the biodiesel or diesel/biodiesel blend in an amount sufficient to reduce the cold filter plugging point of the biodiesel or diesel/biodiesel by at least about 1° C., or by at least about 2, 3, 4, 5, 6, 7, 8, 9, 10, 11, 12, 13, 14 or 15° C., or by about 1 to abut 20° C., or about 1 to 10, 1 to 5, 2 to 20, 5 to 20, 10 to 20, 2 to 10, 5 to 10 or 5 to 15° C., e.g. by about 1, 2, 3, 4, 5, 6, 7, 8, 9, 10, 11, 12, 13, 14, 15, 16, 17, 18, 19 or 20° C.

The present invention also provides a process for preparing an additive for a biodiesel fuel or for a diesel/biodiesel blend, comprising combining a polymer having a comb structure and one or more saccharide esters, wherein the one or more saccharide esters comprise both saturated and unsaturated ester groups. Thus the additive may be preformed and then added to the biodiesel or blend. Alternatively, the components of the additive may be added separately to the biodiesel or blend, or some may be combined and some added separately to the biodiesel or blend. In each case, the blending should be such that the polymer and saccharide(s) are in the biodiesel or blend in the required amounts. These amounts, for one particular additive composition, are:
Polymer: 0.91% w/w or w/v
Sucrose myristate: 0.06%
Sucrose oleate: 0.03%

The process may comprise the step of determining from the nature of the biodiesel fuel or diesel/biodiesel blend a preferred polymer to be used in the additive. It may comprise determining from the nature of the biodiesel fuel or diesel/biodiesel blend a preferred chain length of ester groups in said polymer. It may comprise synthesising the polymer. It may comprise synthesising the polymer using carboxylic acids and/or carboxylate esters having the determined preferred chain length. The process may comprise the step of determining from the nature of the biodiesel fuel or diesel/biodiesel blend a preferred saccharide ester or mixture of saccharide esters to be used in the additive. It may comprise determining from the nature of the biodiesel fuel or diesel/biodiesel blend a preferred chain length of ester groups in said saccharide ester or mixture of saccharide esters. It may comprise synthesising the saccharide ester or mixture of saccharide esters. It may comprise synthesising the saccharide ester or mixture of saccharide esters using carboxylic acids and/or carboxylate esters having the determined preferred chain length.

In some embodiments the additive comprises some diesel or biodiesel. In this case the additive without the diesel or biodiesel may be prepared as described above, and this may then be mixed with the diesel or biodiesel so as to produce the additive. This mixture may be homogenised as described above. The combining may be such that the final additive has a diesel or biodiesel to polymer ratio of between about 0.5 and 5 to 1, or about 0.5 and 2, 0.5 and 1, 1 and 5, 2 and 5 or 1 and 3, e.g. about 0.5, 0.75, 1, 1.5, 2, 2.5, 3, 3.5, 4, 4.5 or 5 to 1 on a weight basis.

Following addition of the additive to the biodiesel or blend to form an initial mixture, either as a preformed additive or as components, the initial mixture may be homogenised. This may comprise heating and or agitating. The heating may be to a suitable temperature for dissolution and/or homogenisation, e.g. about 30 to 60° C., or about 30 to 40, 40 to 50 or 50 to 60° C., e.g. about 30, 35, 40, 45, 50, 55 or 60° C. The agitation may comprise shaking, stirring, sonicating, swirling or a combination of these.

The saccharide esters may be made by esterifying the corresponding saccharides. This may be achieved by reacting the saccharide with a suitable acid halide (e.g. acid chloride) or mixture of acid halides. The ratio of saccharide to acid halide should be approximately the same as the desired substitution ratio. Thus for example if sucrose-5-oleate is to be prepared, the mole ratio of sucrose to oleyl chloride would be about 1:5; if sucrose-2-oleate-3-myristate is to be prepared, the ratio of sucrose to oleyl chloride to myristyl chloride would be about 1:2:3 etc. The reaction may be conducted in a suitable solvent of combination of solvents. Commonly the saccharide will be dissolved in a dipolar aprotic solvent, such as DMF, DMSO, pyridine, HPMT, HMPA etc. This may require heating the mixture of saccharide and solvent. A catalyst, for example a tertiary amine, is then added to the resulting solution. In the event that the solvent is a tertiary amine, this step may of course be omitted. Suitable catalysts include pyridine, triethylamine, DMAP or other known catalysts for this reaction. Prior to the addition of the catalyst, the solution may be cooled, for example to below the boiling point of the catalyst, in order to reduce evaporative loss of the catalyst. The acid halide is then added. It may be added neat or in a solvent, for example acetonitrile, chloroform, DMF, acetone, dichloromethane, dichloroethane or other suitable aprotic solvent. After reaction at a suitable temperature for a suitable time (e.g. about 1 hour at 60° C., or 0.5 to 2 hours at about 40 to 80° C., or other suitable conditions as may be readily determined), the reaction mixture is commonly cooled and treated with a carbonate or bicarbonate (e.g. sodium or potassium carbonate or bicarbonate). Prior to and during reaction of the acid halide with the saccharide, the solvents and reagents should be dry in order to minimise hydrolysis of the acid halide. Thus the process may comprise the step of drying one or more of the solvents and reagents used.

It will be understood that other methods of esterification of saccharides may be used, and these are generally known.

The polymers may be made by copolymerising the corresponding monomers. This may be achieved by free radical polymerisation. Thus the monomers may be combined, preferably dissolved, in a solvent. The nature of the solvent will be dependent on the nature of the monomers. A suitable solvent for many instances is toluene, however others, such as xylene or mixed solvents, may also be used. Commonly the resulting solution is deoxygenated. This may be achieved by purging with an inert gas (e.g. nitrogen, argon, helium, neon etc.). Alternatively it may be by degassing, e.g. using freeze-thaw cycles (commonly at least 2, optionally 3 or 4 cycles). A free radical initiator is added to the solution, either before or after the deoxygenation. Suitable initiators are well known, and may include peroxides, hydroperoxides, peroxyesters, azo initiators etc. The resulting solution should be heated to a temperature suitable for thermal decomposition of the initiator in order to generate initiating radicals. This temperature will depend on the half-life of the initiator. It may be the 10 hour half-life temperature of the initiator, or the 5, 2, 1, 0.5, 0.2 or 0.1 half-life of the initiator, or some other suitable temperature. In this context the x hour half-life temperature is defined as that temperature at which the half-life of the initiator is x hours. The polymer may be isolated from the reaction mixture by commonly known methods, for example precipitation, solvent evaporation etc. It may be dried e.g. in air, optionally with heating, in order to remove residual solvent.

### BRIEF DESCRIPTION OF THE DRAWINGS

Preferred embodiments of the present invention will now be described, by way of example only, with reference to the accompanying drawings wherein:

FIG. 1 shows structures of acrylic acid, methacrylic acid and maleic anhydride olefinic monomers used in comb polymers (P1-P8) including an example of a terpolymer repeat unit;

FIG. 2 shows an overlay of DSC results obtained for BDT01DV (neat biodiesel), 1% w/w Span 85 in BDT01DV and 1% w/w Span 65 in BDT01DV;

FIG. 3 shows changes in CFPP and CP by increasing % w/w additives in B20

FIG. 4 shows changes in CP and CFPP with increasing sucrose myristate (SMy) content within the internal ratio of surfactants @ 1.75% w/w (1.66/0.088);

FIG. 5 shows changes in CP and CFPP with increasing ester to P4 content @ 1.75% w/w in B20;

FIG. 6 shows the effect of varying ratio of polymer:esters on the CFPP of the Blends CFPP of various additive levels in B20; and

FIG. 7 shows CFPP of various biodiesel blends with different optimized additive package.

### DETAILED DESCRIPTION OF THE PREFERRED EMBODIMENTS

### Abbreviations

Abbreviations used in the following description include the following:
Bx (e.g. B10) Biodiesel/diesel blend (x % biodiesel)
BDT25 DV tallow based biodiesel
CFPP cold filter plugging point
CP cloud point
DA isodecyl acrylate
DiBM dibutyl maleate
DSC differential scanning calorimetry
LA lauryl acrylate
LM lauryl methacrylate
MA maleic anhydride
NATA National Association of Testing Authorities
PEG polyethylene glycol
SA stearyl acrylate
SM stearyl methacrylate
SMy sucrose myristate
SO sucrose oleate
Tol toluene
VCBD Victoria Chemicals biodiesel

The cold flow properties of biodiesel and conventional petrodiesel are extremely important. Unlike gasoline, petrodiesel and biodiesel can both start to freeze or gel as the temperature gets colder. If the fuel begins to gel, it can clog filters or can eventually become too thick to pump from the fuel tank to the engine. The Cold Filter Plugging Point (CFPP) is the temperature at which fuel crystals have agglomerated in sufficient amounts to cause a test filter to plug. The CFPP is less conservative than the cloud point (CP), and is considered by some to be a better indication of low temperature operability.

CFPP was analysed on a Herzog HCP842, which complies with ASTM 6371-05 Standard Test Method for Cold Filter Plugging Point for Diesel and Heating Fuelsor on an ISL brand CFPP analyser which is NATA certified using the relevant ASTM or ISO method

### Preparation of Biodiesel/Additive Mixtures for DSC and CFPP

The majority of additives were tested from 0 to 3.5% w/w concentration in beef/mutton biodiesel. To prepare a biodiesel/additive mixture, the desired biodiesel/petrodiesel blend was combined with the additive (about 1.575 g) in a Kartell 50 ml storage bottle. This mixture was then warmed on a hot plate equipped with a magnetic stirrer. The sample was heated to 60° C. and stirred for 15 minutes, then removed from the heat and shaken vigorously, or sonicated to homogenise the contents.

### Polymer Additives

### (Representative Procedure for Poly(Alkyl Acrylate-Alkyl Methacrylate-Maleic Anhydride Terpolymers)

P4—LA/SM/MA (see below): A 250 mL, 2-necked round bottom flask equipped with a stirrer and purged with nitrogen was charged with lauryl acrylate (0.04 mol) and stearyl methacrylate (0.008 mol) dissolved in 50 mL of toluene. Maleic anhydride (1.96 g; 0.02 mol) was then added to the vigorously stirring solution and toluene (15 mL) was used to wash down the inside of the reaction flask. The temperature was increased to 65-75° C. prior to adding benzoyl peroxide (0.24 g), using, if necessary, a minimum amount of toluene to wash down the inside of the reaction flask. After 3 hours, an additional amount of benzoyl peroxide (0.24 g) was added and the temperature increased to 80-85° C. After another 3 hours, the heat was turned off and the reaction mixture left to stir overnight. Isolation of the polymer was achieved by precipitation into vigorously stirring methanol (400 mL). The polymer was collected by decanting off approx. 300 mL of solution and then winding the polymer onto a plastic spatula. The polymer was placed into a teflon petri dish, covered loosely with aluminium foil and placed in the oven overnight to drive off any residual solvent.

### Comb Polymers (P1-P8 Inclusive)

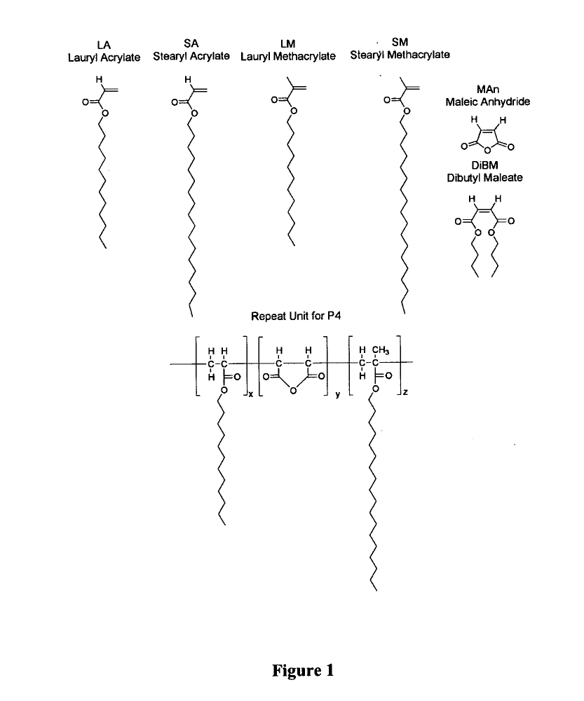

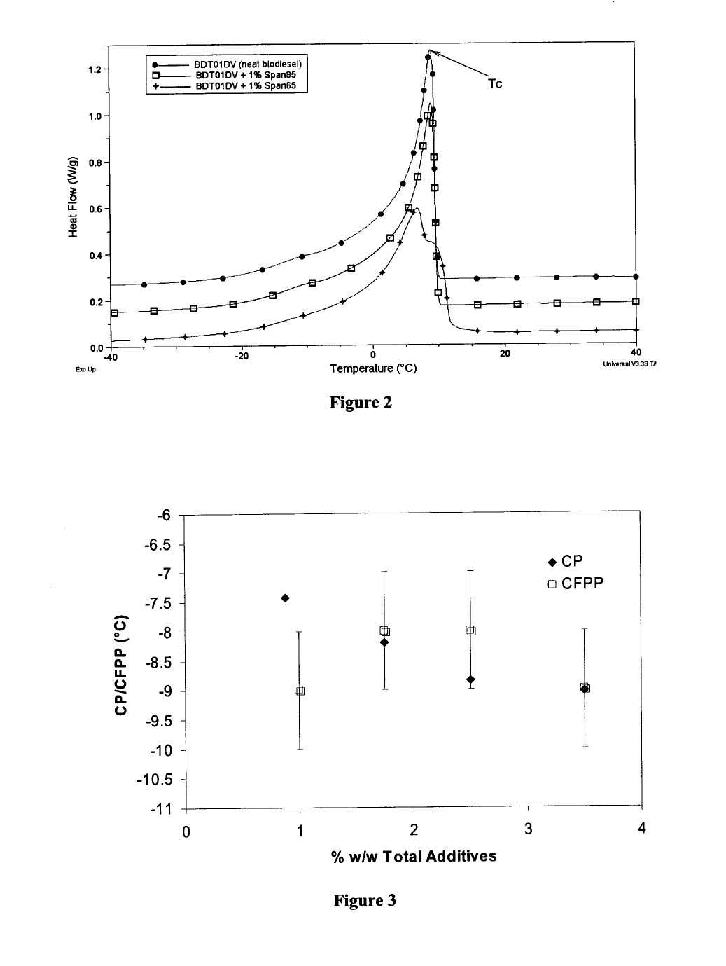

Eight different polymers (FIG. 1, Table 1) were prepared by polymerizing varying mole ratios of acrylate, methacrylate and maleic anhydride olefinic monomers under free radical forming conditions. The polymerization was conducted by heating a mixture of the monomers and free radical initiator dissolved in the desired solvent at a temperature range between 65-85° C. under inert atmosphere. After approximately 6 hours the heat was turned off and the reaction mixture allowed to return to room temperature and left to stir overnight. The polymer was isolated by precipitation into vigorously stirring methanol, collected and placed in an oven overnight to remove any residual solvent.

TABLE 1
Polymer names and mole ratios of monomers used in the synthesis
of P1-P8 inclusive and their relative Tₒₙₛₑₜ values.

| | | Mole | Tₒₙₛₑₜ^{(a)} |
|---|---|---|---|
| Polymer | Monomers | Ratios | (° C.) |
| P1 | SA/LM/DiBM | 2:0.5:1 | 9.91 |
| P2 | LA/SM/DiBM | 2:0.5:1 | 8.54 |
| P3 | SA/LM/M | 2:0.5:1 | 9.27 |
| P4 | LA/SM/MA | 2:0.5:1 | 7.59 |
| P5 | SA/SM/MA | 2:0.5:1 | 10.00 |
| P6 | LA/MA | 2:1 | 8.16 |
| P7^{(b)} | LA/SM/MA | 2:0.5:1 | 7.66 |
| P8 | LA/LM/MA | 2:0.5:1 | 9.47 |

^{(a)}1.75% polymer/1.75% toluene in tallow-based biodiesel BDT01DV (CP 10.21° C.);
^{(b)}double the amount of benzoyl peroxide used compared to P4.

Based on the Tₒₙₛₑₜ data, the following observations were made regarding the combination of monomers in the terpolymers and their subsequent effectiveness at lowering the cloud point of tallow-based biodiesel:
C18 acrylate (SA) was less effective than C12 acrylate (LM);
Variation in chain length between the acrylate and methacrylate monomers produced more favourable results than when the chain lengths were the same length (e.g. P4 versus P8 and P3 versus P5);
Maleic anhydride monomer was more effective than dibutyl maleate monomer;
Of all the monomer combinations tried, P4 was found to be the most effective polymer-based additive at lowering the cloud point and CFPP of tallow-based biodiesel.

### Surfactants

Surfactants are commonly used at low concentrations in commercial biodiesel additive packages to modify the size and/or shape of the crystals formed. In order to select the best surfactant for inclusion in polymer/biodiesel formulations a total of twelve ‘purchased/commercial’ surfactants and five ‘synthesised’ surfactants were assessed by DSC and CFPP at 1% w/w concentration in biodiesel. Many of these surfactants did not dissolve well in biodiesel (without warming or the use of a solvent) as a result of their high polarity. In fact, the surfactants tested could be classified into two groups: those that dissolved and those that did not. Interestingly, there were significant differences between the effects of soluble and insoluble surfactants on the crystallisation behaviour of biodiesel. Tween 80, PGPR (at 1% w/w), sucrose laurate and sucrose myristate and/or oleate were the only surfactants to lower the cloud point of biodiesel outside of experimental error limits. Of these surfactants only PGPR was soluble in biodiesel, however as for Lactem PQ 25K it has a short shelf life. The cloud point results alone suggest that it would be prudent to use Tween 80 and the sucrose esters in polymer/biodiesel/surfactant formulation studies. However sucrose myristate was selected for further investigation in biodiesel/petrodiesel blends as it displays the additional benefit of lowering the saturated enthalpy of crystallisation to a greater degree than the other cloud point-lowering surfactants.

### (Representative Procedure for Sucrose Ester Synthesis)

Sucrose Myristate (5 equivalents): Sucrose (1.71 g, 0.005 mol) and dry DMF (5 mL) were heated in a 3 neck 250 mL round bottomed flask (equipped with a condenser, thermometer and pressure equalising dropping funnel) with a heat gun until solution was achieved. Dry pyridine (0.03 mol, 2.86 g, 2.43 mL) was added and the solution cooled to 60° C. A solution of myristoyl chloride (0.025 mol, 6.17 g, 6.8 mL) in CH₃CN (5 mL) and acetone (10 mL) was added dropwise to the vigorously stirred solution over a 30 min period (when making sucrose palmitate, stearate or behenate, the solvent was dichloromethane, dichloroethane or chloroform). After the addition of the acid chloride the solution was stirred at 60° C. for 1 hour, cooled to room temperature and acetone (75 mL) added. Sodium bicarbonate (2.5 g) water (0.125 mL) were added to the flask to decompose the pyridine hydrochloride by-product. After the evolution of CO₂ ceased, Na₂SO₄ to remove water and mixture filtered through celite on a sintered funnel. At this point if the solution is brown, charcoal can be added to decolourise. The solvent removed by high vacuum distillation (water bath 45° C.) and dried on high vacuum to afford the desired mixture as an off-white solid (7.71 g, 98%). Analysis of the mixture by chromatographic means was impossible so bulk analysis by ¹H NMR afforded the ratio of sucrose to fatty acid. This was achieved through the silylation of the sucrose ester with BSTFA (N,O-bis(trimethylsilyl)trifluoroacetamide) in CHCl₃ solution, evaporation and removal of excess BSTFA by drying on high vacuum (0.1 mmHg) NMR ratio of TMS (trimethylsilyl)ethers to terminal methyl signal of fatty acid residue was calculated via integration and gave a total of 5 fatty acid esters present in the mixture. It should be stressed that this value is an average value, and that species were present with more or less than 5 fatty acid esters per molecule.

### Alternative Method for Synthesis of Sucrose-5-Myristate and Sucrose-5-Oleate.

Sucrose (1.71 g, 0.005 mol) and dry DMF (5 ml) were heated in a 3 neck 250 mL round bottomed flask (equipped with a condenser, thermometer and pressure equalising dropping funnel) with a heat gun until solution was achieved. Dry pyridine (0.03 mol, 2.86 g, 2.43 ml) was added and the solution cooled to 60° C. A solution of myristoyl chloride (0.025 mol, 6.17 g, 6.8 ml) or oleoyl chloride (0.025 mol, 7.52 g, 8.27 ml) in acetone (15 ml) was added dropwise to the vigorously stirred solution over a 30 min period. After the addition of the acid chloride the solution was stirred at 60° C. for 1 hour, cooled to room temperature and acetone (75 ml) added. Sodium bicarbonate (2.5 g) and water (0.125 mL) were added to the flask to decompose the pyridine hydrochloride by-product. After the evolution of CO₂ ceased, Na₂SO₄ to remove water and mixture filtered through a sintered (G3 or G4) funnel. The solvent removed by rotary evaporation, then high vacuum distillation (water bath 45° C.) before drying on high vacuum. The average yield was 7.6 g (sucrose-5-myristate) or 8.5 g (sucrose-5-oleate).

### DSC Results of Polymer/Toluene/Surfactant Blends in BDT01DV

Table 5 below presents a full description of the incorporation of surfactants, as well as favoured polymer P4, into unmodified biodiesel. Surfactants were commonly added at 5% of the overall additive mixture, usually blended with toluene at 50/50% w/w, with the remaining 95% comprising the polymer and toluene in equal amounts. Some variations with the concentration of the surfactant set at 10% of overall additive mixture were also trialled.

TABLE 5
cloud point, T_{c} and ΔTₛₐₜᵤᵣₐₜₑₛ values for surfactant/biodiesel mixtures

| | Additive | Cloud | | |
|---|---|---|---|---|
| | Concentration | Point | ΔTₛₐₜᵤᵣₐₜₑₛ | ΔTₛₐₜᵤᵣₐₜₑₛ |
| Additive/Biodiesel Mixture | (% w/w) | (° C.) | (° C.) | (% w/w) |
| BDT01DV | | 10.21 | | |
| Toluene/BDT01DV | 3.5 | 8.58 | 1.63 | 0.466 |
| P4T/BDT01DV | 0.5 | 9.66 | 0.55 | 1.10 |
| P4T/BDT01DV | 3.5 | 7.59 | 2.62 | 0.749 |
| 95% P4T&5% SMT/DT01DV | 3.5 | 7.67 | 2.54 | 0.726 |
| 95% P4T&5% SM(NoTol)/BDT01DV | 3.5 | 7.65 | 2.56 | 0.731 |
| 95% P4T&5% SMSOT/BDT01DV | 3.5 | 8.29 | 1.92 | 0.549 |
| 95% P4T&5% | 3.5 | 6.84 | 3.37 | 0.963 |
| Span85:Tol[50:50 w/w])/BDT01DV# | | | | |
| 95% P4T&5% | 3.5 | 7.97 | 2.24 | 0.640 |
| Span85:Tol[50:50 w/w])/BDT01DV# | | | | |
| 95% P4T&5% SST/BDT01DV# | 3.5 | 7.82 | 2.39 | 0.683 |
| 95% P4T&5% SST/BDT01DV# | 3.5 | 7.95 | 2.26 | 0.646 |
| 95% P4T&5% GeminiSurfactant: | 3.5 | 8.13 | 2.08 | 0.594 |
| Tol[50:50 w/w]/BDT01DV | | | | |

#= Sample made 2 times with each having different concentrations of polymer/toluene/surfactant (shown in Table 10)
P4T = P4:Toluene (50/50% w/w)
SMyT = Sucrose Myristate:Toluene (50/50% w/w)
SMySOT = Sucrose Myristate:Sucrose Oleate:Toluene (25/25/50% w/w)
SST = Span 65:Span 85:Toluene (25/25/50% w/w)
S85T = Span85:Toluene (50:50% w/w)

A typical co-additive procedure was to create separate 50/50 mixtures of Polymer/Solvent and Surfactant/Solvent. These two entities were then usually blended in the ratio of 95/5 (Polymer in Toluene/Surfactant in Toluene) to give a total weight fraction of 3.5% additive in Biodiesel.

### CFPP Results of Polymer/Toluene/Surfactant Blends in VCBD

TABLE 6

| Additive/ | Additive | Cloud | | | |
|---|---|---|---|---|---|
| Biodiesel | Concentration | Point | CFPP | ΔTₛₐₜᵤᵣₐₜₑₛ | ΔTₛₐₜᵤᵣₐₜₑₛ |
| Mixture | (% w/w) | (° C.) | (° C.) | (° C.) | (% w/w) |
| VCBD | — | 8.44 | 10 | | |
| P4T/VCBD | 0.5 | 7.50 | 8 | 0.94 | 1.88 |
| 95% P4T&5% | 0.5 | 7.26 | 8 | 1.18 | 2.36 |
| SMyT/VCBD | | | | | |
| 95% P4T&5% | 0.5 | 7.44 | 8 | 1.00 | 2.00 |
| SST/VCBD | | | | | |
| 95% P4T&5% | 0.5 | 7.33 | 7 | 1.11 | 2.22 |
| SMySOT/ | | | | | |
| VCBD | | | | | |
| P4T/VCBD | 3.5 | 5.95 | 6 | 2.49 | 0.711 |
| 90% P4T&10% | 3.5 | 6.00 | 7 | 2.44 | 0.697 |
| SMyT/VCBD | | | | | |
| 95% P4T&5% | 3.5 | 5.76 | 6-7 | 2.68 | 0.766 |
| SMyT/VCBD | | | | | |
| 95% P4T&5% | 3.5 | 5.84 | 7-8 | 2.60 | 0.743 |
| SMySOT/ | | | | | |
| VCBD | | | | | |
| 95% P4T&5% | 3.5 | 6.70 | 5-6 | 1.74 | 0.497 |
| SST/VCBD | | | | | |

P4T = P4:Toluene (50/50% w/w),
SMyT = Sucrose Myristate:Toluene (50/50% w/w),
SMySOT = Sucrose Myristate:Sucrose Oleate:Toluene (25/25/50% w/w),
SST = Span 65:Span 85:Toluene (25/25/50% w/w)

When the CFPP was run with normal biodiesel/petrodiesel blends with P4 (no surfactants), at the CFPP temperature (the temperature at which the measurement fails to suck up 20 ml of biodiesel in 60 seconds) the sample usually stayed in the pipette because the filter was blocked and frozen over. When the surfactants were added, once the CFPP temperature was reached and the test stopped, all of the sample collected in the 20 ml pipette flowed back into the containment vessel, indicating that the size of the crystals were smaller and were only just large enough to disallow passage through the filter, but, not large enough to block the filter when the pressure was reduced and gravity takes over. The best result came from the combination of P4 with the Span 65 & Span 85 combination of surfactants at 3.5% total additive mixture. Adding the surfactants in at lower concentrations seems to be the trend in current systems. The surfactant, sucrose myristate was then tried at two different combinations: 5% and 10% of 3.5% additive overall. At the higher concentration (10%) it returned both a higher CP and CFPP. At the lower concentration (5%) different surfactants were trialled: sucrose myristate, sucrose myristate, sucrose oleate, Span 65 and Span 85.

### SUMMARY

The use of polymers in isolation generates a strong correlation between CFPP and CP. This indicates that these parameters are strongly coupled. Terpolymers such as P4, have been found to have a strong influence upon CP, and correspondingly on CFPP. When surfactants are added CFPP begins to decouple from CP, and added benefits arise. Two surfactant packages, Span 65/Span 85 and sucrose myristate/sucrose oleate were identified as promising candidates. The benefit achieved with CFPP generally ranges from 4-5° C.

Previously no work has been done using fatty acid sucrose esters as surfactants for biodiesel additives. Through the use of molecular modelling, it was shown sucrose esters having no free hydroxy groups (i.e. sucrose octaester) had decreased surfactant properties and tends to cause aggregation in a much similar fashion to that of methyl stearate within biodiesel. Thus it was proposed to use specific equivalents of the acid chloride reactant in the synthesis of the sucrose ester, as shown below.

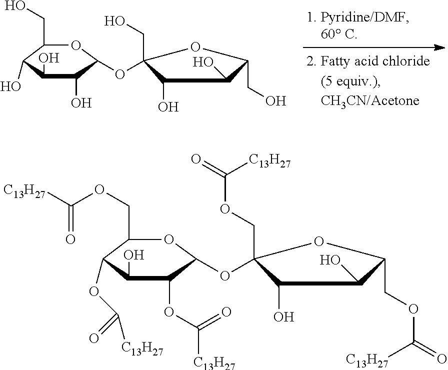

### Synthesis of Sucrose Ester Surfactants (Sucrose Myristate—5 Equivalents)

### Optimisation

The diesel specification testing results for the 3.5% w/w package (unoptimised) below. This package comprise a ratio of 2:1 (w/w) polymer:esters. The esters were sucrose myristate and sucrose oleate in a weight ratio of 1:1 and the polymer was a terpolymer of lauryl acrylate, stearyl methacrylate and maleic anhydride in a molar ratio of 2:0.5:1. are detailed in Table 7. The original additive met the diesel specification for the following parameters; Sulphur, Ash and suspended solids, Cetane Index, Density, Viscosity, Water and sediment, Conductivity at ambient temperature, Oxidation stability, Colour, Copper corrosion, Filter Blocking Tendency, Lubricity and Distillation (95% recovered).

TABLE 7
Diesel Specification Testing of 3.5% w/w additive (unoptimised)

| | Fuel Standard | | 3.5% w/w |
|---|---|---|---|
| | (Automotive | | unoptimised |
| Property | Diesel) | | additive package |
| Sulphur | 500 | ppm (max) | 14 |
| Ash and Suspended | 100 | mg/kg (max) | 33.4 |
| Solids | | | |
| Cetane Index | 46 | (min) | 48.3 |
| Density | 820 to 860 | kg/m³ | 836.8 |
| Viscosity | 2 to 4.5 | cSt @ 40° C. | 2.783 |
| Carbon Residue (10% | 0.2% | mass (max) | 0.6 |
| Distillation residue) | | | |
| Water & Sediment | 0.05% | vol (max) | 0.01 |
| Conductivity @ | 50 pS/m (min) @ | | 1179 @ 19.2° C. |
| Ambient Temp. | ambient temp. | | |
| Oxidation Stability | 25 | mg/L (max) | 2 |
| Colour | 2 | max | 1 |
| Acidity | Total: 0.08 mg | | 1.16 |
| | KOH/g (max) | | |
| Copper Corrosion | Class 1 (max) | | 1 |
| (3 hrs @ 50° C.) | | | |
| Flash Point | 61.5° | C. (min) | 61.5° C. |
| Filter Blocking Tendency | 2 | (max) | 1.62 |
| Lubricity | 0.460 | mm (max) | 0.194 |
| Distillation - 95% | 370° | C. (max) | 338.7° C. |
| Recovered | | | |

| | | |
|---|---|---|
| Cloud Point ** | — | −3° C. |
| Cold Filter Plugging | — | −10° C. |
| Point (CFPP) ** | | |

CP and CFPP Sample Preparation of Blends and Blends with Additives

The unmodified additives package as developed in above was prepared at 3.5% in toluene and tested in Victoria Chemicals biodiesel (VCBD), B20 made from VCBD and Caltex diesel, and Caltex diesel. All biodiesel/diesel blends described herein were prepared on a weight basis. To prepare additive samples for analysis in DSC and CFPP, toluene was removed from the additives package as diesel was sufficient in dissolving the polymer and ester components. The total additives package used in comparisons for optimisation of the package in B20 was 1.75% w/w. The ratio of Polymer:Esters:B20 was 1:0.0526:59.1. The ratio of ester co-surfactants was 1:1, i.e. 1:0.0263:0.0263:59.1

To make CFPP and DSC(CP) samples, each component of the package was dissolved in B20 then mixed in a Kartell 50 ml storage bottle, or a small sample vial (10 g). This mixture was then warmed on a hot plate. The sample was heated to 60° C. and removed from the heat and shaken vigorously to homogenise the contents. CFPP samples were also made by addition of all contents into a Schott bottle with the addition of B20 and subsequently sonicated at 50° C. for 30 minutes.

### Test Procedure Used in DSC:

Temperature calibration and cell constant=high purity indium

### Baseline=−90° C. to 90° C.

Sample size=5.0±0.5 mg
Pan type=Aluminium Hermetic (TA Instruments)
Heating rate=5° C./min
Gas flow rate and composition=50 ml/min high purity nitrogen (BOC Gases)
Scan parameters=1) equilibrate at 80° C., 2) isothermal for 1 min, 3) ramp 80° C. to −80° C., 4) isothermal for 1 min, 5) ramp −80° C. to 80° C.

### Reduction in Cloud Point

This parameter is used to evaluate whether the additive has an effect on the saturated onset temperature of crystallisation (cloud point) of a blend sample. It is calculated according to Equation 1.1:

Tₒₙₛₑₜsaturates(B20)−Tₒₙₛₑₜsaturates(B20+additive)

Comparison of Fuels with Additives Package

Initially, the additives package including toluene at 3.5% w/w was tested in B100, Caltex Diesel and B20 made from Caltex. In preparation of B20 with additives, no toluene was needed to dissolve the additives in the fuel, as the additives dissolved in diesel. This reduced the total additives package to 1.75% w/w. The CFPP and CP (as tested by DSC) of these initial tests are given in Table 8. Tests at this ratio were unable to be performed in B100 due to the insolubility of the additives in B100.

TABLE 8
CFPP and CP data for Unmodified Additives Package (AP) at 3.5% and
1.75% w/w

| | CFPP | CFPP + | CFPP + | CP | CP + | CP + |
|---|---|---|---|---|---|---|
| Sample | ° C. | 3.5% AP | 1.75% AP | ° C. | 3.5% AP | 1.75% AP |
| B100 | 8 | 5 | — | 8.4 | 5.4 | — |
| Caltex | −3 | −8 | −7 | −6.22 | −9.64 | −9.07 |
| Diesel | | | | | | |
| B20 | −2 | −10 | −8 | −4 | −9.68 | −8.2 |

### Polymer

Due to the high cost associated with using lauryl acrylate, the substitution of this monomer with isodecyl acrylate (DA) and lauryl methacrylate (LM) was investigated. Two new polymers were prepared using the same method outlined previously. The incorporation of DA/SM/MA and LM/SM/MA polymers into the additives package at 1.75 wt % in B20 was shown to increase the cloud point of B20 by approximately 2° C. (Table 10).

TABLE 10
Mole ratios of monomers used in the synthesis of P4,
DA/SM/MA and LM/SM/MA polymers and their DSC results.

| | Mole ratio of monomers | | | |
|---|---|---|---|---|
| | Lauryl | Stearyl | Maleic | CP |
| | acrylate | methacrylate | anhydride | In B20 + |
| Polymer | (LA) | (SM) | (MA) | Esters |
| P4 | 0.04 | 0.008 | 0.02 | −8.13 |
| LA/SM/MA | | | | |
| DA/SM/MA | 0.04 | 0.008 | 0.02 | −5.97 |
| LM/SM/MA | 0.04 | 0.008 | 0.02 | −5.56 |

The use of diesel and mixed xylenes as polymerisation solvents was investigated using the same synthetic procedure as described earlier. The use of diesel as a reaction solvent was unsuccessful as maleic anhydride is insoluble in diesel. The polymerisation conducted in mixed xylenes was successful. The cloud points obtained by DSC for the polymers made in toluene and mixed xylenes are comparable, as seen in Table 11. Therefore, mixed xylenes are an alternative solvent for preparation of P4.

TABLE 11
Mole ratios of monomers used in the synthesis of
P4 in different solvents and their cloud points.

| | Mole ratio of monomers | | | |
|---|---|---|---|---|
| | Lauryl | Stearyl | Maleic | CP |
| | acrylate | methacrylate | anhydride | In B20 + |
| Polymer | (LA) | (SM) | (MA) | Esters |
| P4 (new) made | 0.04 | 0.008 | 0.02 | −8.13 |
| in toluene | | | | |
| P4 made in | 0.04 | 0.008 | 0.02 | −8.33 |
| xylene | | | | |

In order to optimise the internal ratio of the monomers, the amount of LA and/or MA was altered. The ratios investigated are displayed in Table 12 and were synthesised based on the procedure described previously. Increasing the amount of MA in the reaction mixture (P4-1), increased the cloud point by approximately 5° C., thus, further increases were not investigated. Decreasing the amount of MA (P4-3), exhibited a slight decrease in CP. Further reduction of MA was not investigated due to the cost increase associated with the polymer. Decreasing the amount of LA by 20% (P4-6), 50% (P4-7) and 60% (P4-8), exhibited decreases in the cloud points, with the most significant decrease obtained at a 50% reduction in LA.

TABLE 12
Mole ratios of monomers used and their DSC results.

| | Mole ratios of monomers | | | |
|---|---|---|---|---|
| | Lauryl | Stearyl | Maleic | CP |
| | acrylate | methacrylate | anhydride | In B20 + |
| Polymer | (LA) | (SM) | (MA) | Esters |
| P4 (new) | 0.04 | 0.008 | 0.02 | −8.13 |
| P4-1 (+15% MA) | 0.04 | 0.008 | 0.023 | −2.9 |
| P4-3 (−15% MA) | 0.04 | 0.008 | 0.017 | −8.7 |
| P4-6 (−20% LA) | 0.032 | 0.008 | 0.02 | −8.61 |
| P4-7 (−50% LA) | 0.02 | 0.008 | 0.02 | −9.47 |
| | | | | −9.06 |
| P4-8 (−60% LA) | 0.016 | 0.008 | 0.02 | −8.94 |

The synthetic procedure as presented earlier was followed with removal of the solvent by rotary evaporation. After rotary evaporation, the polymer was placed in the oven overnight at 80° C. in order to ensure completion of the polymerisation. The DSC is results of the polymers obtained with no precipitation were comparable to that of the polymer made by the unmodified method. An increase in yield was also obtained using this improved method. This new method is a considerable improvement for the synthesis of comb polymers both in terms of cost and yield.

In order to decrease the cost of the polymer synthesis, a series of experiments were conducted where the concentrations of benzoyl peroxide and solvent were varied. The polymerisation time was also varied between 1 and 5 hours. The reaction conditions, yield and DSC cloud point results are summarised in Table 13. The synthetic procedure was further modified in experiment No 21, where the starting materials were mixed at 60° C. for 10 min to allow the materials to dissolve as it had been observed that maleic anhydride did not dissolve in toluene at room temperature. The reaction mixture was then poured into a jar and left in the oven overnight at 80° C. ¹H NMR of the polymer product confirmed that polymerisation was complete. The procedure used in exp 22 was additionally simplified where all starting compounds were mixed in a jar at 60° C. for 10 minutes using hot plate and then left overnight in the oven at 80° C. (Note that oven was connected to nitrogen flow.)

TABLE 13
Synthesis of P4 varying the reaction conditions

| | | | DSC | | |
|---|---|---|---|---|---|
| Exp | | | (in B20 + | | |
| No | Polymer | Yield | Esters) | NMR | FTIR |
| 1 | P4 | 84 | −8.21 | ✓ | ✓ |
| 2 | P4 A (2X BP 2 hrs, prec) | 80 | −8.2 | ✓ | ✓ |
| 3 | P4 B (½ BP, 2 hrs, prec) | 72 | −8.65 | ✓ | |
| 4 | P4 C(½ BP 5 hrs, prec) | 70 | −8.55 | ✓ | |
| 5 | P4 D (½ BP 2 hrs, prec) | 72 | −8.33 | ✓ | |
| 6 | P4 E(½ BP 2 hrs, no prec) | Cross-linked | | | |
| 7 | P4 E (repeated) | 75 | −8.25 | ✓ | |
| 8 | P4 F(½ BP 2 hrs, prec | No polymer; heated to 80° C. | | | |
| | 60° C. | | | | |
| 9 | P4 G (1/4 BP 2 hrs, no prec) | 96 | −7.85 | ✓ | |
| 10 | P4 H (½ BP 1 hrs, no prec) | Cross-linked | | | |
| 11 | P4 H (½ BP 1 hr, no prec) | 98 | −7.35 | ✓ | |
| | (repeated) | | | | |
| 12 | P4 I (¼ BP 1 hr, no prec) | 98 | Cross-linked | ✓ | |
| | | | 2^{nd} heating | | |
| 13 | P4 I 1(¼ BP 1 hr, no prec) | 98 | Cross-linked | ✓ | |
| | repeated | | 2^{nd} heating | | |
| 14 | P4 I 2 (¼ BP 1 hr, no prec) | 98 | Cross-linked | ✓ | |
| | repeated | | 2^{nd} heating | | |
| 15 | P4 I 3(¼ BP 1 hr, no prec) | 98 | Cross-linked | ✓ | |
| | repeated | | 2^{nd} heating | | |
| 16 | P4 I 4(¼ BP 1 hr, no prec) | 98 | Cross-linked | ✓ | |
| | repeated | | 2^{nd} heating | | |
| 17 | P4 J 1(½ BP 5 hrs, ½ Tol, | 98 | −7.73 | ✓ | |
| | no prec) | | | | |
| 18 | P4 J 2 (½ BP 3 hrs, ½ Tol, | 98 | −7.55 | ✓ | |
| | no prec) | | | | |
| 19 | P4 K 1 (¼ BP 5 hrs, ½ Tol, | 97 | −8.42 | ✓ | |
| | no prec) | | | | |
| 20 | P4 K2 (¼ BP 3 hrs, ½ Tol, | 99 | −7.58 | ✓ | |
| | no prec) | | | | |
| 21 | P4 L (¼ BP 1 hr, ¼ Tol, | 98 | −7.8 | ✓ | |
| | stirred at 60, polymerised in | | | | |
| | oven) | | | | |
| 22 | P4 L 1 (¼ BP 1 hr, ¼ Tol, | 99 | −8.03 | ✓ | |
| | stirred at 60 in jar, | | | | |
| | polymerised in oven) | | | | |

The above improvements were incorporated into an improved synthetic method, described below. P4, P4-6 and P4-7 were prepared in triplicate using the improved method.

Maleic anhydride, lauryl acrylate, stearyl methacrylate and toluene (10 ml) were introduced into a small jar containing a magnetic stirrer. The reaction mix was placed on a hot plate at approximately 60° C. and stirred until maleic anhydride was completely dissolved. Benzoyl peroxide was added and the remaining (2 ml) toluene was used to wash down the benzoyl peroxide and added into the jar. After the mixture was homogenous, the jar was placed in the oven overnight. A small stream of nitrogen gas was used to purge the oven, while the temperature of the oven was set at 80-90° C. ¹H NMR and FTIR of each triplicate were performed and confirm the new polymerisation method is reproducible. The yield, DSC and CFPP data are compared for the triplicates in Table 14. P4 (optimised synthesis), P4-6 (optimised synthesis and 20% less Lauryl acrylate) and P4-7 (optimised synthesis and 50% less Lauryl acrylate) were tested with the unmodified ester ratios at the current concentration of additives at 1.75% w/w in B20. It can be seen from Table 14, that the CFPP data of the different polymers is comparable. The cloud point results from DSC again suggest that P4-7 has a lower cloud point than P4 or P4-6, as previously discussed.

TABLE 14
Triplicate reactions with yields, CP and CFPP

| | Mole ratios of monomers | | | | | | | CP | |
|---|---|---|---|---|---|---|---|---|---|
| | Lauryl | | Stearyl | | Maleic | | | In | CFPP In |
| | acrylate | | methacrylate | | anhydride | | | B20 + | B20 + |
| Polymer | (LA) | | (SM) | | (MA) | | Yield (%) | Esters | Esters |
| P4 rxn 1 | 0.04 | mol | 0.008 | mol | 0.02 | mol | 99.4 | −8.2 | −10 |
| | 9.62 | g | 2.71 | g | 1.96 | g | | | |
| | 10.88 | ml | 3.13 | ml | | | | | |
| P4 rxn 2 | 0.04 | mol | 0.008 | mol | 0.02 | mol | 99.5 | −8.04 | −8 |
| | 9.62 | g | 2.71 | g | 1.96 | g | | | |
| | 10.88 | ml | 3.13 | ml | | | | | |
| P4 rxn 3 | 0.04 | mol | 0.008 | mol | 0.02 | mol | 99.3 | | −8 |
| | 9.62 | g | 2.71 | g | 1.96 | g | | | |
| | 10.88 | ml | 3.13 | ml | | | | | |
| P4-6 | 0.032 | mol | 0.008 | mol | 0.02 | mol | 99.5 | −7.98 | −8 |
| rxn 1 | 7.69 | g | 2.71 | g | 1.96 | g | | | |
| | 8.7 | ml | 3.13 | ml | | | | | |
| P4-6 rxn 2 | 0.032 | mol | 0.008 | mol | 0.02 | mol | 100 | | −8 |
| | 7.69 | g | 2.71 | g | 1.96 | g | | | |
| | 8.7 | ml | 3.13 | ml | | | | | |
| P4-6 rxn 3 | 0.032 | mol | 0.008 | mol | 0.02 | mol | 99.2 | | −8 |
| | 7.69 | g | 2.71 | g | 1.96 | g | | | |
| | 8.7 | ml | 3.13 | ml | | | | | |
| P4-7 rxn 1 | 0.02 | mol | 0.008 | mol | 0.02 | mol | 99.3 | | −10 |
| | 4.81 | g | 2.71 | g | 1.96 | g | | | |
| | 5.44 | ml | 3.13 | ml | | | | | |
| P4-7 rxn 2 | 0.02 | mol | 0.008 | mol | 0.02 | mol | 99.4 | −8.89 | −8 |
| | 4.81 | g | 2.71 | g | 1.96 | g | | | |
| | 5.44 | ml | 3.13 | ml | | | | | |
| P4-7 rxn 3 | 0.02 | mol | 0.008 | mol | 0.02 | mol | 99.4 | | −8 |
| | 4.81 | g | 2.71 | g | 1.96 | g | | | |
| | 5.44 | ml | 3.13 | ml | | | | | |

### Changing Additive Concentration in B20

Changes in CFPP and CP by increasing the percent total additives in B20 are shown in FIG. 3. There appears to be no significant difference in CFPP, which ranges from −8 to −9° C. with an increasing additive content. The CP as determined by DSC shows a gradual decrease in temperature with an increase in total additives.

### Ratio of Surfactants

All tests were performed in B20 and made from Caltex diesel on a weight basis. The total additives package was kept constant at 1.75% w/w. This is composed of 1.66% Polymer and 0.088% Esters. The ester component (surfactants) is 0.044% sucrose myristate and 0.044% sucrose oleate in the unmodified additives package.

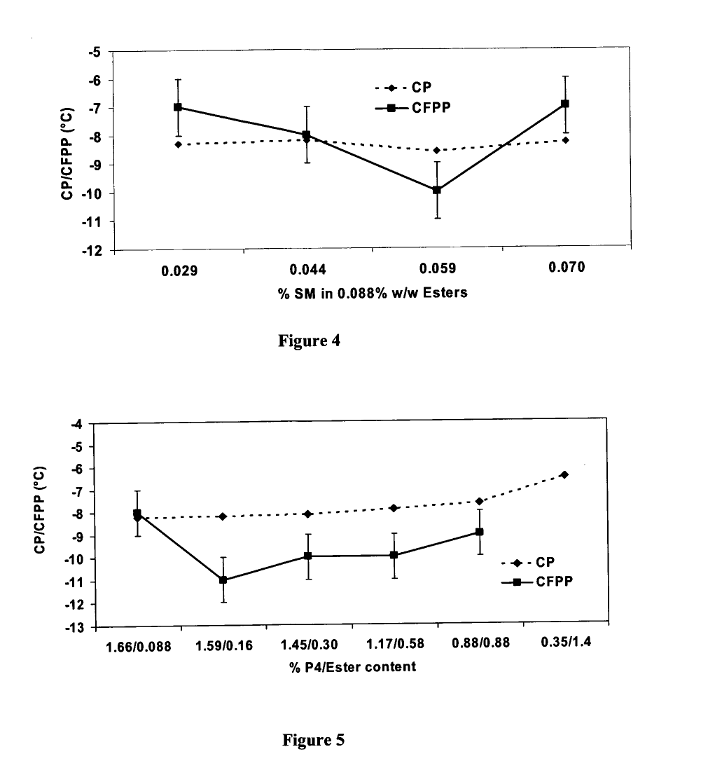

The ratio of sucrose myristate to sucrose oleate was systematically changed while keeping the same ratio of esters to polymer and total additives of 1.75% w/w (esters+polymer). The CFPP and CP data generated are shown in FIG. 4.

The current ratio of SMy:SO is 0.044%:0.044% w/w (i.e. 1:1 w/w). As can be seen in FIG. 5, there appears to be a limiting ratio of SMy to SO where the CFPP value is at its lowest. This occurs at 0.059% SMy/0.029% SO (2:1) and gives a CFPP of −10° C. There was no significant change in cloud point by changing the internal ratios of the surfactants. It therefore appears that the optimum ratio of Sucrose Myristate to Sucrose Oleate was 0.059% to 0.029% (2:1) in the current additives package (1.75 w/w=1.66% Polymer: 0.088% Esters).

### Ratio of Polymer to Surfactant

The total additives package was kept constant at 1.75% w/w in B20. The unmodified ratio of Polymer to Esters is 1.66% to 0.088% (0.044% SO:0.044% SMy). The ratio of polymer to esters was systematically changed with the aim of finding the best CFPP result. These results are shown in FIG. 5.

As may be seen in FIG. 5, the best CFPP result at −11° C. was achieved at a Polymer:Ester ratio of 1.59%/0.16% w/w. The limiting ratio of Polymer:Ester is achieved at 1.17%/0.58%, which gives a CFPP result of −10° C. There is no significant difference in the CFPP from the ratio of 1.59%/0.16% w/w to 1.17%/0.58% Polymer to Esters. The cloud point results increase slightly in temperature with a limiting ratio of 1:1 (0.88%:0.88%) of polymer to esters. Further increasing the ratio of esters led to an increase in the cloud point. Thus it appears that the optimum ratio of Polymer to Esters was found at 1.59%/0.16% w/w (1:0.1) using a total additives package of 1.75% w/w in B20 giving a CFPP of −11±1° C. and a CP of −8° C.

### Optimised Package in B20

It appears from the above that the optimum ratio of Polymer to Esters ranges from 1:0.1 to 1:0.5 and the optimum ratio of co-surfactants is 2:1 sucrose myristate:sucrose oleate. An investigation into the best performing additives packages was conducted by using P4-7 (optimised synthesis and 50% less Lauryl Acrylate) and a combination of the Polymer to Ester ratios and the optimum ratio of co-surfactants.

The first package considered (Optimised package 1) contained a ratio of Polymer:Esters of 1:0.5, with 2:1 SMy:SO. Therefore, the ratio of Polymer:Sucrose Myristate:Sucrose Oleate required was 1:0.33:0.167 at 1.75% w/w and 1% w/w in B20 It can be seen in Table 15, that the CFPP of optimised package 1 was reduced compared to the optimised synthesis with unmodified esters at 1.75% w/w. The CFPP of optimised package 1 at 1% w/w was comparable to that of the optimised synthesis with unmodified esters at 1.75% w/w. Increasing the ester component of the package (optimised package 1) at 1.75% w/w led to an increase in both performance and cost compared to that of the original ester ratio (optimised synthesis with unmodified esters at 1.75% w/w). However, increasing the ester ratio and reducing the additive amount to 1% w/w led to comparable performance with a significant reduction in cost due to the lower additive concentration. The costs are only representative values and should not be included

TABLE 15
CFPP/CP and Cost summary of optimised packages

| | CFPP | CP |
|---|---|---|
| Package | (° C.) | (° C.) |
| PSHIP.191 | −10 | −8.5 |
| 1.75% Optimised package 1 | −11 | −7.63 |
| 1% Optimised package 1 | −8/−8/−7 | −7.42 |
| 1.75% Optimised package 2 | −9 | −8.13 |
| 1% Optimised Package 2 | −9 | −8.1 |
| 1.75% Optimised Synthesis | −8 | −8.9 |
| unmodified esters | | |
| 1% Optimised synthesis + | −11 | −8 |
| unmodified esters | | |
| 1.75% Optimised Synthesis + | −10 | −8.6 |
| 2:1 SMy:SO | | |
| 1% Optimised Synthesis + 2:1 | — | — |
| SMy:SO | | |
| (optimised package 3) | | |

It can be seen that 1% w/w of the optimised package 1 in B20 gives a CFPP result of −11 and performs better than the unoptimised package in particulates and flash point.

The second package considered (optimised package 2) contained a ratio of Polymer to Esters of 1:0.1, with a co-surfactant ratio of 2:1 SMy:SO. The ratio of Polymer:Sucrose myristate: Sucrose Oleate was 1:0.07:0.035 at 1.75% w/w and 1% w/w in B20 This was chosen in order to simultaneously reduce cost by the lower amount of sucrose oleate contained within the additive and increase cold flow performance of the additives package due to the increased total ester content. The CFPP results of optimised package 2 are shown in Table 15 with their comparative costs for 1.75% w/w and 1% w/w. The CFPP of optimised package 2 at 1% w/w is comparable to that of optimised package 1, whilst a reduction of 0.68° C. is evident in the CP of package 2.

### Variation of Blends

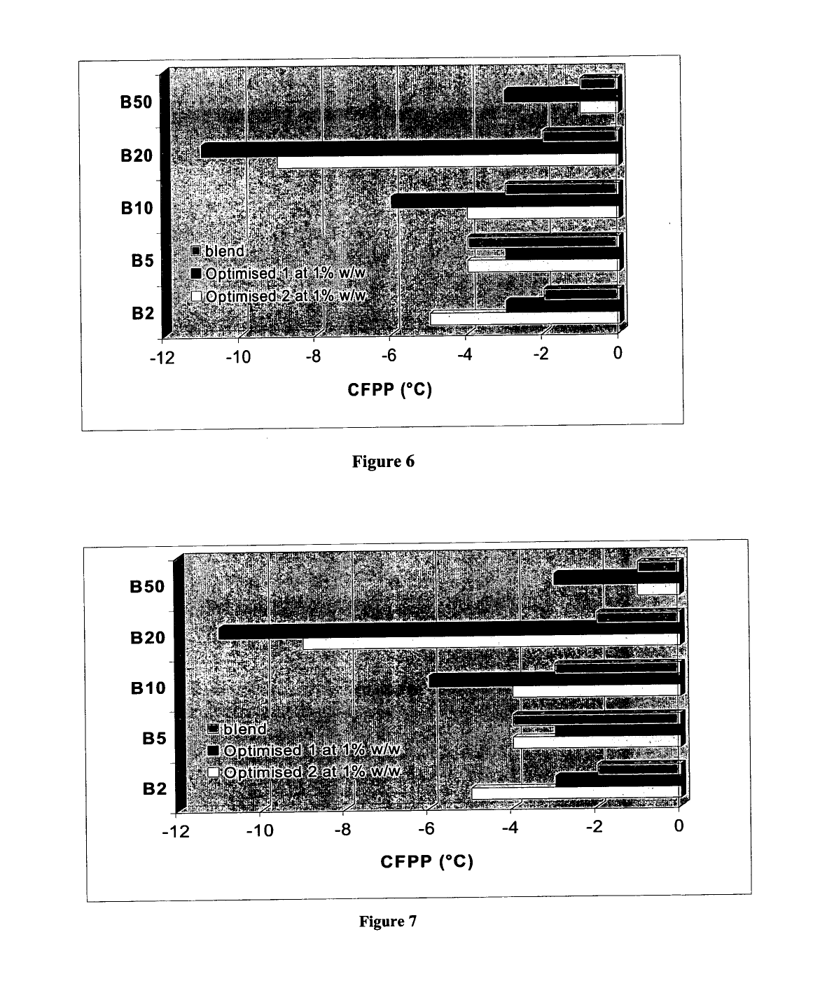

Optimised package 1 and 2 have been tested for CFPP in a variety of diesel blends with the results shown in FIG. 6. A higher ester content (Polymer:Esters 1:0.5 in optimised package 1) produced a greater reduction in CFPP of B10 to B50, while a higher polymer content (Polymer: Esters 1:0.1 in optimised package 2) produced a greater reduction in CFPP of B2 and B5.

B100 used for the blends detailed in this section was produced on a laboratory scale from beef/mutton tallow. The amount of additive used in a B20 blend of Vic Biodiesel used above was reduced from 1.75% to 1% with the CFPP value being −9° C. in both cases. This result is consistent, taking into account the change in feedstock from Victoria Biodiesel to laboratory biodiesel produced from beef/mutton tallow, with that of the beef/mutton B20 with optimized package 2 at 1%, which exhibited a CFPP of −8° C., compared to that of the beef/mutton B20 (summer) of −2° C. Reducing the amount of additive present in the blend is advantageous as this will reduce the total cost of the package, whilst simultaneously reducing the carbon residue and acid number of the subsequent blend, which were identified as problem issues.

### CFPP of Various Additive Levels in B20

The CFPP of a variety of additive levels was investigated from 0% to 1%. An additive level of 0.2% led to a CFPP of −3° C., a reduction in the CFPP of 1° C. Increasing the additive level to 0.4% led to a CFPP of −6° C., a total reduction in CFPP of 4° C. Increasing the additive level to 0.5% and 0.6% exhibited CFPP values of −8° C., a total reduction in CFPP of 6° C. Additive levels of 0.8% and 1% exhibited CFPP values of −9° C. and −8° C., total reductions in CFPP of 7° C. and 6° C., respectively.

### CP of Various Additive Levels in B20

The CP of a variety of additive levels was also investigated from 0% to 1%. An additive level of 0.5% led to a CP of −6.02° C., a reduction in the CP of 2.05° C., whilst increasing the additive level to 0.6% led to a CP of −6.39° C., a total reduction in CP of 2.82° C. Increasing the additive level to 0.8% exhibited a CP of −6.48° C., a total reduction in CP of 2.91° C., whilst an additive levels of 1% exhibited a CP of −6.97° C., a total reduction in CP of 3.4° C.

Similar reductions in additive addition levels were also performed utilizing optimized package 1, the results of which are detailed in Table 16. An additive level of 0.2% led to a CFPP of −3° C., a reduction in the CFPP of 2° C. Increasing the additive level to 0.4% led to a CFPP of −6° C., a total reduction in CFPP of 4° C. Increasing the additive level to 0.5% and 0.6% exhibited CFPP values of −8° C., a total reduction in CFPP of 6° C. Additive levels of 0.8% and 1% exhibited CFPP values of −9° C. and −8° C., total reductions in CFPP of 7° C. and 6° C., respectively.

TABLE 16
CFPP results of various additive levels (OP1) in B20

| % OP1 | CFPP |
|---|---|
| 0.0% | −2 |
| 0.2% | −3 |
| 0.4% | −5 |
| 0.6% | −6 |
| 0.8% | −7 |
| 1.0% | −8 |

Incorporating an additive level of 0.2% led to a CFPP of −3° C., a reduction in CFPP of 1° C. Increasing the additive level to 0.4% led to a CFPP of −5° C., a reduction in CFPP of 3° C. Further increasing the additive level to 0.6% led to a CFPP of −6° C., a reduction in CFPP of 4° C. Additive levels of 0.8% and 1% led to CFPP values of −7° C. and −8° C., total reductions in CFPP values of 5° C. and 6° C., respectively. Comparison of the CFPP to results of OP1 and OP2 indicated that at lower additive levels, OP2 was more effective than OP1, and also more cost effective.

### Fabrication of Optimised Additives Package

The additives package consists of the Polymer and the two Surfactants at the ratios outlined in Table 17.

TABLE 17
Objective Ratio of Polymer to Esters at 1% w/w total additives in the chosen fuel

| | Optimised Package 1 | Optimised Package 2 | Optimised Package 3 |
|---|---|---|---|
| Component ratio | 1% total additive | 1% total additive | 1% total additive |
| Polymer | 1 | 1 | 1 |
| Sucrose-5-Myristate | 0.333 | 0.07 | 0.0351 |
| Sucrose-5-Oleate | 0.167 | 0.0351 | 0.0175 |
| Fuel | 148.5 | 109.41 | 104.21 |

Optimised package 3 wasn't discussed

### Fabrication of the Additives Package in Diesel, or a Biodiesel/Diesel Blend

1. Polymer is warmed to approximately 60° C. in order to lower viscosity for transfer to the desired mixing vessel
2. Sucrose Myristate is a solid and weighed at ambient temperature
3. Sucrose Oleate is a liquid and weighed at ambient temperature
4. All components are mixed at the desired ratio
5. The components are added directly to the fuel (if it is diesel or a diesel blend)
6. Homogenisation of the entire fuel mixture with additives is required (performed by heat and vigorous shaking or sonication at a laboratory scale)
7. In scale-up, it is envisaged the additive will be dissolved and homogenised in a fraction of the total fuel, which is then transferred to the bulk fuel and subsequently mixed.

### Example of Additive Calculation for a Fuel

BASIS: 1000 kg of FUEL (diesel or blend)
Assumption: density of additives is=to density of fuel
Ratio used: 1% w/w with 2:1 SMy: SO=1:0.0351:0.0175:104.21

### Total=105.26

### Polymer=1000/105.26=9.5 kg

### Sucrose Myristate=9.5*0.0351=333.45 g

### Sucrose Oleate=9.5*0.0175=166.25 g

### Fuel=9.5*104.21=989.99 kg

Total=(9.5+0.33345+0.16625+989.999)=999.999 kg (tidy up significant figures to ensure it equals 1000 kg?)
CP and CFPP Sample Preparation of Blends and Blends with Additives (this Needs to Address the Fact that the Feedstock was Varied Here)

Optimised additives package 2, as described above was prepared and tested at 1% w/w in biodiesel, made in the laboratory from tallow supplied from Midfields, B20 made from biodiesel and Caltex (summer) or Mobil (winter) diesel. All biodiesel/diesel blends described herein were prepared on a weight basis. The ratio of Polymer:Esters:B20 of optimized package 2 is 1:0.1052: X, where X is the amount of fuel required, which varies depending on the blend used. The ratio of ester co-surfactants was 1:1, i.e. 1:0.0701:0.0351: X.

To make CFPP and DSC(CP) samples, each component of the package was dissolved in B20 then mixed in a Schott bottle with the addition of B20 and subsequently sonicated at 50° C. for 30 minutes.

### CFPP Results

The summer diesel used for blending in this section of the work exhibited a CFPP of −3° C., with a CP, of −6.22° C., whilst winter diesel exhibited a CFPP of −6° C., with a CP of −9.27° C. (have summer and winter diesel been discussed previously, if not that should go into the introduction) Beef biodiesel was blended with summer diesel to B20, with the blend exhibiting a CFPP of −2° C. The additives package developed in the previous stage of the project, herein described as optimized package 2, was incorporated into the B20 (summer) blend at a 1% w/w level, with the subsequent blend yielding a CFPP of −8° C. Beef B100 was also blended with winter diesel to B20, with this blend exhibiting a CFPP of −5° C. Incorporation of optimized package 2 at a 1% w/w level into the B20 (winter) blend gave rise to a CFPP of −7° C. The CFPP data for the summer and winter diesel blends with the various feedstocks and additive are summarized in Table 18.

Mutton biodiesel was blended with summer diesel to B20, resulting in a CFPP of −1° C. Optimized package 2 was incorporated into the B20 (summer) blend at a 1% w/w level, with the resultant blend exhibiting a CFPP of −9° C. Mutton B100 was also blended with winter diesel to B20, with this blend exhibiting a CFPP of −3° C. Incorporation of optimized package 2 at a 1% w/w level into the B20 (winter) blend exhibited a CFPP of −7° C.

Beef/mutton biodiesel was blended with summer diesel to B20, with the blend resulting in a CFPP of −2° C. Optimized package 2 was incorporated into the B20 (summer) blend at a 1% w/w level, with the resultant blend yielding a CFPP of −8° C. Beef/mutton B100 was also blended with winter diesel to B20, with this blend exhibiting a CFPP of −5° C. Incorporation of optimized package 2 at a 1% w/w level into the B20 (winter) blend exhibited a CFPP of −10° C.

TABLE 18
CFPP results (° C.) of B20 blends
with winter and summer diesels and additive

| | | B20C + | | B20M + |
|---|---|---|---|---|
| | B20C | OP2 1% | B20M | OP2 1% |
| Beef | −2 | −8 | −5 | −7 |
| Mutton | −1 | −9 | −3 | −7 |
| Beef/Mutton | −2 | −8 | −5 | −10 |

### CP Results

Beef biodiesel was blended with summer diesel to B20, with the blend exhibiting a CP of −2.66° C. The additives package developed in the previous stage of the project, herein described as optimized package 2, was incorporated into the B20 (summer) blend at a 1% w/w level, with the subsequent blend yielding a CP of −6.58° C. Beef B100 was also blended with winter diesel to B20, with this blend exhibiting a CP of −3.6° C. Incorporation of optimized package 2 at a 1% w/w level into the B20 (winter) blend gave rise to a CP of −8.04° C. The CP data for the summer and winter diesel blends with the various feedstocks and additive are summarized in Table 19.

Mutton biodiesel was blended with summer diesel to B20, resulting in a CP of −1.10° C. Optimized package 2 was incorporated into the B20 (summer) blend at a 1% w/w level, with the resultant blend exhibiting a CP of −6.36° C. Mutton B100 was also blended with winter diesel to B20, with this blend exhibiting a CP of −2.6° C. Incorporation of optimized package 2 at a 1% w/w level into the B20 (winter) blend exhibited a CP of −5.41° C.

Beef/mutton biodiesel was blended with summer diesel to B20, with the blend resulting in a CP of −3.57° C. Optimized package 2 was incorporated into the B20 (summer) blend at a 1% w/w level, with the resultant blend yielding a CP of −6.97° C. Beef/mutton B100 was also blended with winter diesel to B20, with this blend exhibiting a CP of −4.73° C. Incorporation of optimized package 2 at a 1% w/w level into the B20 (winter) blend exhibited a CFPP of −6.03° C.

TABLE 19
CP results (° C.) of B20 blends
with winter and summer diesels and additive

| | | B20C + | | B20M + |
|---|---|---|---|---|
| | B20C CP | OP2 1% CP | B20M CP | OP2 1% CP |
| Beef | −2.66 | −6.58 | −3.6 | −8.04 |
| Mutton | 1.57 | −6.35 | −2.6 | −5.51 |
| Beef/Mutton | −3.57 | −6.97 | −4.73 | −6.03 |

### Conclusions

B100 from various tallow feedstocks exhibited comparable CP and CFPP results
Significant decreases, between 10 and 19° C., in CFPP and CP were evident up blending of biodiesel from the various feedstocks with both summer and winter diesels.
The incorporation of optimised package 2 into the B20 blends of biodiesel from the various feedstocks with summer diesel, exhibited decreases in CP and CFPP from 1 to 8° C.
Decreases from 3 to 5° C. were evident in the CP and CFPP of winter diesel B20 blends incorporating optimized package 2.
Additives package is effective when the % saturates exceeds the % unsaturates
Employment of palmitate side chains in the additive could increase the additive effectiveness with palm (and possibly tallow) blends.

### Final Optimised Additive Package

The additive package consists of a specialised copolymer and sucrose surfactants The ratios can vary with the blend used as detailed in FIG. 7.

The copolymer comprises the following:

Maleic anhydride

Lauryl Acrylate

Stearyl Methacrylate

These monomers are in an internal ratio of 1:2:0.5
The sucrose surfactants are:

Sucrose Oleate

Sucrose Myristate

These are in a ratio of Sucrose Myristate to Sucrose Oleate of 2:1
The polymer to surfactant ratio is 1:0.1

### Surfactant

It was shown that extended side chain length substitutions of the sucrose 5 myristate showed no measurable difference in CP and CFPP. The optimum internal ratio of sucrose myristate to sucrose oleate determined to be 2:1.

### Polymer

Mixed xylene was found to be an alternative solvent for polymerization of the copolymer, giving a further reduction in cost. Modification of the polymer through the use of different chain lengths, such as isodecyl acrylate and lauryl methacrylate increased the cloud point. A 50% reduction in the lauryl acrylate component yielded the best CP and CFPP results. The cloud point was also increased by changing the internal monomer ratios. The optimum ratio of polymers to esters ranged from 1:0.1 to 1:0.5.

From the fine tuning of the polymer and surfactant components, the best optimised additives package at 1% w/w in B20 consisted of
Polymer to ester ratio from 1:0.1 to 1:0.5
Co-surfactant ratio of 2:1 Sucrose Myristate to Sucrose Oleate
   This is depicted in FIG. 7 which details the CP and CFPP results for each blend and two different optimised packages, with package 1 (OP1 1:0.5) and package 2 (OP2 1:0.1). Optimised package 2 had a lower cost due to the reduction in surfactant component.

1. An additive for lowering the minimum usable temperature of a biodiesel fuel or a diesel/biodiesel blend, said additive comprising:
at least one saccharide ester, and
a polymer having a comb structure,
wherein:
if only one saccharide ester is present, said saccharide ester comprises at least one saturated ester group and at least one unsaturated ester group; and
if more than one saccharide ester is present, these comprise a first saccharide ester comprising at least one saturated ester group and a second saccharide ester comprising at least one unsaturated ester group.

2-5. (canceled)

6. The additive of claim 1 wherein the ratio of the polymer to the at least one saccharide ester is between about 5:1 and about 20:1 w/w.

7. The additive of claim 1 wherein the polymer is a maleic anhydride derived copolymer.

8. The additive of claim 3 wherein the polymer is a maleic anhydride/acrylate ester/methacrylate ester derived terpolymer.

9. The additive of claim 1 wherein the polymer comprises side chains derived from lauric acid, side chains derived from stearic acid or both of these.

10-11. (canceled)

12. The additive of claim 1 wherein the saccharide ester, or both the first and second saccharide esters, have an average of about 3 to about 7 ester groups per molecule.

13. The additive of claim 1 wherein the saccharide ester, or at least one of the first and second saccharide esters, is a disaccharide ester.

14. The additive of claim 7 wherein the disaccharide ester is a sucrose ester.

15. The additive of claim 1 wherein only one saccharide ester is present, said ester being a sucrose myristate oleate mixed ester.

16. The additive of claim 1 comprising a first saccharide ester and a second saccharide ester wherein the first saccharide ester is sucrose myristate and the second saccharide ester is sucrose oleate.

17. The additive of claim 1 wherein the ratio of saturated esters to unsaturated esters is about 2:1 on a weight or molar basis.

18. The additive of claim 1 wherein the length of a carbon chain in an ester group of the saccharide ester(s) and/or a length of a side chain of the polymer is matched to the length of a carbon chain in the biodiesel.

19. (canceled)

20. A biodiesel or diesel/biodiesel blend comprising an additive according to claim 1.

21. The biodiesel or diesel/biodiesel blend of claim 13 wherein the additive is present in the biodiesel or diesel/biodiesel blend at between about 0.1 and about 3.5% on a w/w, w/v or v/v basis.

22. The biodiesel or diesel/biodiesel blend of claim 13 wherein said additive is present in the biodiesel fuel or diesel/biodiesel blend at less than about 3.5% w/w and wherein the cold filter plugging point of said biodiesel or diesel/biodiesel blend is reduced by at least about 2° C. relative to said biodiesel or diesel/biodiesel blend without said additive.

23. A process for preparing an additive for lowering the minimum usable temperature a biodiesel fuel or a diesel/biodiesel blend, said process comprising combining a polymer having a comb structure and at least one saccharide ester, wherein:
if only one saccharide ester is used, said saccharide ester comprises at least one saturated ester group and at least one unsaturated ester group; and
if more than one saccharide ester is used, these comprise a first saccharide ester comprising at least one saturated ester group and a second saccharide ester comprising at least one unsaturated ester group.

24. The process of claim 18 wherein the polymer and the at least one saccharide ester are combined in a ratio of between about 5:1 and about 20:1 w/w.

25. A method for reducing the minimum usable temperature of a biodiesel fuel or of a diesel/biodiesel blend, said method comprising combining said biodiesel fuel or diesel/biodiesel blend with an additive according to claim 1.

26. The method of claim 20 wherein the additive is combined with the biodiesel fuel or diesel/biodiesel blend at a ratio of between about 0.1 and about 3.5% on a w/w basis.

27-28. (canceled)

29. The biodiesel or diesel/biodiesel blend of claim 13 wherein the biodiesel comprises an animal derived biodiesel.

30. The biodiesel or diesel/biodiesel blend of claim 13 wherein the biodiesel comprises a vegetable derived biodiesel.
